(19) United States
(12) Patent Application Publication
(10) Pub. No.: US 2006/0099628 A1
(43) Pub. Date: May 11, 2006

(54) Diagnostic assay for rickettsia prowazekii disease by detection of responsive gene expression

(76) Inventors: Wei-Mei Ching, Bethesda, MD (US); Hong Ge, Potomac, MD (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/272,419

(22) Filed: Nov. 9, 2005

Related U.S. Application Data
(60) Provisional application No. 60/627,811, filed on Nov. 10, 2004.

(51) Int. Cl. C12Q 1/68; G06F 19/00

(52) U.S. Cl. 435/6; 702/20

(57) ABSTRACT

The inventive subject matter relates to a method for the detection and diagnosis of Rickettsia prowazekii infection by measuring the increased or decreased expression of specific human genes following infection by microarray or polymerase chain reaction analysis. Gene modulation profiles can be further analyzed by computer. The method permits the early detection and diagnosis of Rickettsia prowazekii exposure and infection and diagnosis of epidemic typhus earlier than any currently available methods.

### CROSS-REFERENCES TO RELATED APPLICATIONS

This application claims priority to U.S. Provisional application 60/627,811 filed Nov. 10, 2004.

### BACKGROUND OF THE INVENTION

1. Field of the Invention

The inventive subject matter relates to a method of diagnosing epidemic typhus caused by the bacteria Rickettsia prowazekii by analysis of modulation of host gene expression. The method contemplates the use of microarray technology for the detection and analysis of gene up or down regulation in response to bacterial infection.

2. Description of the Related Art

Rickettsia prowazekii, the etiologic agent of epidemic typhus is characterized as a short, rod-shaped gram-negative bacteria. Human infection by the bacteria is via infected louse feces. In addition to causing epidemic typhus, R. prowazekii is also the causative agent of recrudescent typhus (Brill-Zinsser disease), a reactivation of latent infection typically occurring in areas of high R. prowazekii endemicity. Worldwide, the disease is associated with significant mortality. The disease is generally associated with a lack or disruption of adequate medical infrastructure and hygiene caused by war, famine and social disruption (1). For example, during world war I, more than 3 million people are estimated to have died and 25 million people to have been infected (2).

Epidemic typhus is also an important health issue in regions where the organism is highly endemic, primarily mountainous regions of Mexico and Guatemala, the Andes of South America, the Himalayan countries (e.g. Pakistan and Afganistan), the highland regions of Africa (e.g. Ethiopia, Burundi, Rwanda, and Lesotho) and Northern China. A major epidemic in 1997 infected 100,000 people in Burundi (3-5). Furthermore, epidemic typhus is a re-emerging infectious disease in Africa, South America and Eastern Europe. A recent survey indicated that 20% of the rural Andean population was infected with R. prowazekii (6). Due to frequent and often long-lasting civil unrest in areas of the world where the organism is endemic a high risk of typhus epidemics exists to the area inhabitants. Also, travelers to these areas are at significant risk of exposure to the organism and contracting epidemic typhus.

In addition to naturally acquired infection, because of its relative availability, R. prowazekii has the potential for use as a bio-terrorism agent. Furthermore, since R. prowazekii is stable in dried louse feces, the normal transmission vector, the disease is capable of being disseminated in aerosol form thus affecting large numbers (1, 7). Additionally, the organism can be transported long distances in dried feces and used to contaminate targeted food or water supplies.

Infection with drug resistant strains, following exposure by naturally endemic organisms or through purposeful human dissemination as a bio-terrorism agent, would multiply mortality significantly. R. prowazekii is typically highly resistant to all but two classes of commonly used antibiotics, tetracycline and chloramphenicol (8,9). Recently, a rifampin resistant strain of R. prowazekii was made by electroporation of a plasmid containing the target mutation (10). Resistance to chloramphenicol and tetracycline could be similarly achieved and create a significant bio-terrorism threat agent.

A number of diseases are caused by closely related Rickettsiae bacterial species. Differential diagnosis is important to design specific treatment regimens that may be effective against one organism but not against others, such as the case in epidemic typhus. Current methods for the diagnosis of rickettsial disease, such as epidemic typhus, are most often based on clinical presentation and the exposure history of patients. However, differentiation of R. prowazekii infection from infection by bacteria causing other acute febrile illnesses can be difficult because of the similarities in symptoms.

Available laboratory diagnostic methods for rickettsial diseases includes nucleic acid detection by polymerase chain reaction (PCR) amplification of rickettsial genes (11-13) and serodiagnostic assays such as Weil-Felix, ELISA, Dip-S-Ticks (DS), the indirect immunoperoxidase (IIP) assay and indirect immunofluorescent IFA) tests (14, 15). Although these methods are well established, they lack the sensitivity required for detecting early biological responses resulting from host infection by the bacteria. PCR-based assays aimed at detecting pathogen directly are typically only possible days after infection when adequate DNA template is available. Similarly, antibody-based assays, such as ELISA, typically require even more time to complete than PCR. Antibody based assays designed to detect serum conversion following infection at least one to two weeks to develop an adequate measurable host immune response.

Gene expression modulation, however, unlike other measured parameters, are manifested early following exposure of infectious organisms. Unique gene expression profiles are exhibited early after infection of human peripheral blood mononuclear cells (PBMC) to different pathogens and toxins, including Bacillus anthracis, Yersinia pestis, Brucella melitensis, botulinum toxin, staphylococcal exotoxins A and B (SEB, SEA), lipopolysaccharide (LPS), cholera toxin, Venezuelan equine encephalitis virus (16). A number of genes were previously shown to be modulated following infection following bacterial infection are shown in Table 1 (16, 18, 19).

TABLE 1
Genes up-regulated in response to bacterial infection

| ChemokinesReceptor | Signaling | Apoptosis/Growth Arrest |
|---|---|---|
| CXCL1 | NFKB1 | GADD45 alpha |
| CCL3 | ENG | |
| CCL10 | KYNU | |
| CCL3 | | |
| CCL20 | | |
| MIP1 beta | | |

| Cytokines | Adhesion | Transcription |
|---|---|---|
| IL1B | TNFAIP3 | DSCR1 |
| | TNFAIP6 | |

Semi-quantitative reverse transcriptase polymerase chain reaction (RT-PCR) is capable of sensitively measuring changes in gene expression from collected host cell RNA. Designing primer sets specific to a limited number of genes known have altered expression following infection can be used to diagnosis and monitor infection early after infection.

A more convenient method of measuring gene expression changes is by hybridizing expressed RNA onto cDNA microarrays containing large numbers of double-stranded sequences of important host genes. A number of computer programs are available to accurately analyze and transform the ensuing gene expression data into useful and reproducible gene expression profiles.

Microarrays are well suited for high-throughput detection of thousands of differentially expressed genes in a single experiment (17). The method allows for the characterization of the cascade of cellular signaling and concomitant interrelated host gene expression profiles following infection by specific pathogens or toxins (18, 19). Therefore, data from cDNA microarrays provides the ability to quickly and accurately assess and monitor the changes in gene expression profiles specific to infection by specific pathogenic organisms. Microarrays can also be used to evaluate genomic differences between virulent and nonvirulent strains of a species by comparing the host response to the organisms (20).

Specific human genes have been previously disclosed that modulate up or down in response to bacterial infection (21). Analysis of human gene expression can therefore be a predictor of infection by specific microorganisms. The general approach, therefore, of evaluating changes in human gene expression can be utilized as an effective diagnostic tool very early after infection, when other currently available methods are not effective. The approach can be used alone or in tandem with other methods, therefore to follow progression of the disease state through treatment.

Therefore, in order to improve early diagnosis of epidemic typhus, an aspect of this invention is the diagnosis of R. prowazekii early after exposure and infection by the measurement of host gene expression. Measurement of host gene expression is by any method capable of detecting and quantitatively or semi-quantitatively measuring gene expression, such as semi-quantitative RT-PCR or microarray analysis. The invention, therefore, will permit diagnosticians with the ability to diagnosis R. prowazekii days or weeks earlier than previously possible. Additionally, the care provider will be able to monitor the course of the disease more accurately and therefore the effectiveness of the drug regimen employed.

### SUMMARY OF INVENTION

Current methods for the detection and diagnosis of epidemic typhus, caused by Rickettsia prowazekii early after infection are inadequate. An object of this invention is a method for the early diagnosis of Rickettsia prowazekii hours after exposure and infection to the organism and the monitoring of the disease course by the measurement of the modulation of expression of specific host cell genes.

An additional object of the invention is the early diagnosis of epidemic typhus and recrudescent typhus by analysis of the modulation of specific host cell genes expression.

A further object of the invention is the determination of R. prowazekii exposure and infection by the sensitive detection of host gene expression by reverse transcriptase polymerase chain reaction. Specificity of detection of host gene RNA is enhanced by reducing the background due to amplification of contaminating DNA.

A still further object of the invention is the determination of R. prowazekii exposure and infection by analysis of the modulation of specific host cell gene expression using microarray chips containing sequences of host cell genes.

Additionally, another object of the invention is the determination of R. prowazekii exposure and infection by analysis of the modulation of specific host cells gene expressions using antibody-based assays.

Another object of the invention is a set of specific host genes that are modulated in expression in response to R. prowazekii that can be measured by RT-PCR or by microarray hybridization in the early determination of R. prowazekii exposure and infection and diagnosis of epidemic typhus.

### DETAILED DESCRIPTION OF PREFERRED EMBODIMENTS

Epidemic typhus, caused by the bacteria Rickettsia prowazekii is one of the most common rickettsial diseases causing significant mortality in untreated patients. Because of the relatively high mortality rate in untreated patients, the rising prevalence of drug resistant strains, and the lack of vaccines against the organism, early detection of exposure and infection is increasingly important.

Diagnosis of the disease epidemic typhus caused by R. prowazekii early after exposure of individuals to the bacteria is difficult due to a lack of available assay methods. Current methods for the diagnosis of epidemic typhus rely on detection of serum conversion or detection of bacterial antigen or DNA, which is not possible until long after exposure and infection. Other methods call for the direct detection of the organism, which requires a considerable incubation period following infection.

Analysis of human gene expression profiles has become an increasingly important mode of predicting disease onset and for monitoring disease progression. Following exposure to external insults, such as infectious organisms or toxins, some cellular genes are modulated to increase or decrease their expression. Specific cell perturbations, such as viral or bacterial infections, can result in precise and reproducible gene modulation profiles (21). The current invention capitalizes on this phenomenon by monitoring the expression of specific genes early after exposure of human cells to R. prowazekii. The invention employs sensitive methods for the detection and quantitative assessment of changes in specific gene expression. The resulting gene modulation profile of the infected human cells is computer analyzed. From the gene modulation profile, very early detection of R. prowazekii exposure and infection can be obtained and therefore diagnosis of epidemic or recrudescent typhus can be achieved.

A number of methods can be used to measure gene expression. For example, semi-quantitative reverse transcriptase polymerase chain reaction (RT-PCR) can be used, with primers to specific target gene RNA to evaluate changes in gene expression. Primer sets containing at least one of the primers to the mRNA splice site significantly increases specificity and therefore reliability of diagnosis by reducing the amplification of contaminating DNA. Alternatively, or in addition to RT-PCR, labeled cDNA copies of mRNA from the infected human cells can be exposed to complimentary DNA copies of specific genes attached to glass microarray chips and the bound cDNA quantitated. Use of microarrays permits the convenient analysis of large numbers of genes in a single experiment. Evaluation of gene modulation profiles is conducted by computer program analysis.

In addition to RT-PCR or microarrays, direct detection by ELISA or other antibody-based method, can be used to detect expressed gene protein product. An aspect of this invention is the detection and analysis of the modulation of specific human genes following exposure of the cells to R. prowazekii.

Diagnosis of infection is operationally carried out by initially measuring changes in gene expression in response to infection. Any semi-quantitatively or quantitative procedure can be used to measure changes in expression including methods that are capable of detecting changes in specific host cell mRNA, such as RT-PCR or microarray and methods that detect host gene expressed protein product such as antibody-based assays. However, regardless of the specific method used, the general approach in all methods employs the following steps:
a. obtaining blood cells from patients potentially exposed to R. prowazekii;
b. extracting total RNA or protein extract from the blood cells;
c. measuring gene products of a panel of important host genes by molecular, antibody-based or other methods;
d. normalizing the expression of the important host genes in the potentially infected cells to that in uninfected cells;
e. analyzing the pattern or profile of gene modulation by computer program.

Based on the gene modulation profile, a diagnosis early after exposure and infection is made by comparing the profile detected with that associated with the profile associated with R. prowazekii infection. Since diagnosis of infection is able to be made much earlier than by utilizing other methods, early antibiotic treatment can be instituted. Additionally, regular re-evaluation of expressed genes during disease progression permits real-time evaluation of the effectiveness of the drug treatment regimen and modification of treatment methods, if needed. To more clearly describe the invention, the following examples are given.

### EXAMPLE 1

### Detection of Gene Expression in the Cell Line THP-1 by Hybridization of Gene Products to Microarray Chips

Microarray chips have the advantage of permitting the analysis of expression large numbers of genes in a single assay. Because the chips can be manufactured to contained thousands of gene sequences in replicates, microarray chips allows the direct comparison of the modulation of expression of these genes with time after exposure of cells to specific insults such as infectious organisms or toxins.

A specific example of how the method can be practiced is illustrated by the gene response in a human monocytic cell line following infection with R. prowazekii. The gene modulation pattern observed in the cell line is applicable to what would be expected in collected peripheral blood mononuclear cells (PBMC) collected from patients suspected of prior infection with R. prowazekii.

In this study, human monocytic cells (THP-1) are grown to near confluence and infected with R. prowazekii (Breinl strain). The R. prowazekii used to infect the THP-1 cells is propagated in Vero cell monolayers (African green monkey kidney cells). After five to six days of Vero cell culture, the infected cells are harvested. The bacteria are purified by homogenization of the cells followed by separation through Renografin gradient.

THP-1 cells are collected at 1, 4, 8, 18 and 24 hours after infection. Total RNA from the infected cells are collected and treated with DNAse. Additionally, RNA from uninfected (control) THP-1 cells was also collected. The RNA is labeled by reverse transcribing using oligo dT or random primers in the presence of either Cy3 or Cy5. The labeled cDNA is then exposed to glass chips containing cDNA clones encoding approximately 7,800 human gene sequences. The resulting Tagged Image File Format (TIFF) is then computer analyzed.

The cDNA gene fragments were spotted onto poly-L-lysine-coated slides using an OmniGrid arrayer® (GeneMachines, San Carlos, Calif.). After hybridization, the bound, labeled cDNA was scanned and the image analyzed using a GenePix® (Molecular Devices Corporation, Union City, Calif.) or similar computer program. Normalization of induced expression, following R. prowazekii infection, was conducted by comparing expression of RNA from THP-1 cells to control RNA from uninfected THP-1 cells and subtracting from the spot intensity the background intensity in order to produce a channel-specific value.

An analysis of host genes that are modulated in response to R. prowazekii was conducted using microarray analysis. This approach, unlike other methods, such as quantitative polymerase chain reaction (qPCR), enables the evaluation of expression of an extremely large number of genes, in a single experiment. Table 2 shows a panel of host genes that were shown to either increase or decrease in expression (i.e. repressed) by microarray analysis in response to R. prowazekii infection. In the microarray analysis, a gene was determined to be up-regulation or down-regulated expression if there was ≧1.5 fold increase or ≦0.667 fold decrease expression, respectively, at more than one time point.

A relatively large number of genes were shown by microarray analysis to modulate in expression in response to R. prowazekii yielding a specific gene modulation pattern shown in Table 2. Additionally, Table 3 and Table 4 lists genes that are also listed in Table 2 but that have not previously been shown to modulate in expression in response to infection by any other bacterial organism, including the closely related organism Orientia tsutsugamushi. Table 3 shows genes that increased in expression and Table 4 shows genes that decreased in expression in response to R. prowazekii infection.

Confirmation of gene expression modulation, as demonstrated by microarray analysis, was confirmed by quantitative polymerase chain reaction (qPCR). In these studies, total RNA was obtained according to the TRIzol® method (Invitrogen, Carlsbad, Calif.) followed by treatment with DNAse. Oligo dT or random primers were added to the total RNA and the mixture heated at 70° C. for 5 minutes followed by incubation on ice for 3 minutes. Following the addition of the first strand buffer, dNTP, DTT and reverse transcriptase, the final mixture was incubated at 42° C. for 50 minutes and then shifted to 70° C. for 10 minutes.

After production of a cDNA copy of the RNA, primers to selected target sequences were used to amplify the specific target gene sequences. The primer sets were designed such that at least one primer member of a primer set was complementary to the sequence encoding the splice site of the target mRNA. Targeting of the splice junction ensures that amplification of sequences will not occur via remaining genomic DNA as a template and therefore reduces assay background.

Table 5 shows the results of the qPCR analysis for a number of eukaryotic genes, including some that were found to be specifically induced by R. prowazekii. In Table 5, all genes examined by qPCR were previously shown to increase in expression in response to R. prowazekii by microarray analysis. In the microarray analysis, a gene was determined to be up-regulation or down-regulated expression if there was ≧1.5 fold increase or ≦0.667 fold decrease expression, respectively.

TABLE 2
Gene modulation following R. prowazekii infection

| Induced (131) | Repressed (11) |
|---|---|
| Cytokines (5) | PRG1 |
| IL1B | CA2 |
| IL1RL1 | BMI1 |
| G1P3 | STAT5B |
| PBEF1 | NCF4 |
| Chemokines (8) | PLA2R1 |
| IL8 | RPS15A |
| MIP1B/CCL4L | HIST1H4C |
| CXCL1/GRO1 | MYB |
| CXCL3/GRO3/MIP2B | CITED2 |
| CXCL13/BLC | DHRS9 |
| CCL2/MCP1 | |
| CCL3/MIP1A | |
| CCL20 | |
| Adhesion (9) | |
| ITGA2/CD49B | |
| CDH2 | |
| CDH12 | |
| ROBO1 | |
| CAPN9 | |
| TNFAIP6 | |
| LPXN | |
| PCDH8 | |
| APC | |
| Cytoskeletin/motility (7) | |
| SGCD | |
| COL2A1 | |
| PPL | |
| C8orf1 | |
| ARPC2 | |
| SPRR1B | |
| HPCA | |
| Transporter (6) | |
| AQP4 | |
| KCNS3 | |
| SLC25A10 | |
| CLIC4 | |
| ABCA1 | |
| APOH | |
| Calcium ion binding (7) | |
| EFEMP1 | |
| STC1 | |
| PLEK | |
| S100A8 | |
| S100A9 | |
| S100A12 | |
| DSG2 | |
| Receptor (12) | |
| IL7R | |
| P2RY5 | |
| EDNRB | |
| PTPN2 | |
| PTPRB | |
| RARG | |
| ACVR1 | |
| ENG | |
| SERPINE2 | |
| KLRC3 | |
| FCER1A | |
| LY64 | |
| IL27RA | |
| Signalling (15) | |
| KYNU | |
| MAP3K71P2 | |
| PIK3R2 | |
| RGS1 | |
| PDE4B | |
| ARF5 | |
| TM4SF3 | |
| LTBP2 | |
| CDC42 | |
| ARL4A | |
| RAGE | |
| GEM | |
| HSPA8 | |
| DUSP6 | |
| ECT2 | |
| Transcription (14) | |
| NFKB1A | |
| NRIP1 | |
| TFAP2C | |
| OLIG2 | |
| CREM | |
| Ikaros | |
| KLF2 | |
| POU3F4 | |
| IFI16 | |
| ELK3 | |
| TFEC | |
| CXXC1 | |
| ZNF638 | |
| SNAPC3 | |
| Anti-apoptotic (3) | |
| TNFAIP3 | |
| TNFAIP8 | |
| SNCA | |
| Apoptosis/growth arrest (4) | |
| PRKR | |
| PTEN | |
| GADD45 α | |
| PTHLH | |
| Proliferation (3) | |
| CCNF | |
| MTCP1 | |
| IGFBP3 | |
| Enzyme (20) | |
| INDO | |
| AMPD3 | |
| USP12 | |
| USP32 | |
| UBE2B | |
| SIAT8A | |
| SIAT8E | |
| SIAT1 | |
| ENTPD4 | |
| ACADL | |
| PPP1R14B | |
| COX8A | |
| POLA2 | |
| ADH1B | |
| ACSL1 | |
| CYP2C9 | |
| CYP1B1 | |
| TDO2 | |
| ENTPD4 | |
| B3GALT2 | |
| Miscellaneous (18) | |
| CD83 | |
| CYBB | |
| DLX4 | |
| EGR2 | |
| MAFB | |
| CDR2 | |
| C6orf142 | |
| DEAF1 | |
| RAE1 | |
| BM039 | |
| WBP1 | |
| MALAT1 | |
| PLAU | |
| GJA7 | |

TABLE 3
Genes specifically up-regulated in
response to R. prowazekii infection

| Transcription | Enzyme |
|---|---|
| POU3F4 (1, 4, 8, 18, 24) | PPP1R14B (1, 4, 18) |
| ELK3 (1, 4, 18) | |
| OLIG2 (4, 18) | |

TABLE 4
Genes specifically repressed in expression
in response to R. prowazekii infection

| | | | |
|---|---|---|---|
| CA2 | PRG1 | BMI1 | PLA2R1 |
| HIST1H4F | CITED2 | DHRS9 | |

TABLE 5
Host gene response by qPCR and Microarray analysis

| | ^{a}C_{T} (^{b}X ± SD) and ^{c}Fold Expression Changes (qPCR/microarray) | | | | |
|---|---|---|---|---|---|
| Gene | T1 | T4 | T8 | T18 | T24 |
| ^{d}18S rRNA | 26.16 ± 2.41 | 29.86 ± 0.23 | 23.20 ± 0.10 | 27.03 ± 0.24 | 22.42 ± 0.19 |
| | 25.71 ± 0.20 | 23.64 ± 0.19 | 21.32 ± 0.20 | 25.88 ± 0.19 | 23.58 ± 0.18 |
| GADD45A | 32.96 ± 0.44 | 36.20 ± 0.37 | 32.53 ± 0.26 | 34.96 ± 0.19 | 34.60 ± 0.71 |
| | 33.40 ± 0.20 | 32.62 ± 0.63 | 32.43 ± 0.20 | 36.05 ± 0.40 | 34.14 ± 0.58 |
| | change 1.88/5.54 | change 6.25/5.79 | change 3.43/2.62 | change 4.63/4.92 | change 2.17/2.07 |
| PPP1R14 B | 36.26 ± 1.36 | 38.95 ± 1.37 | 34.40 ± 0.78 | 36.22 ± 0.69 | 33.60 ± 0.35 |
| | 36.32 ± 1.46 | 33.83 ± 0.29 | 34.21 ± 0.29 | 37.40 ± 0.18 | 33.63 ± 0.24 |
| | change 1.42/3.27 | change 2.17/3.82 | change 3.23/1.39 | change 5.01/2.49 | change 2.28/1.39 |
| ENG | 33.64 ± 1.21 | 37.38 ± 0.98 | 31.56 ± 0.02 | 33.56 ± 0.35 | 31.50 ± 0.38 |
| | 33.09 ± 0.30 | 32.48 ± 0.40 | 32.10 ± 1.13 | 33.34 ± 0.37 | 31.28 ± 0.15 |
| | change 0.93/4.75 | change 2.50/4.01 | change 5.35/2.23 | change 1.91/4.16 | change 1.92/1.82 |
| NFKB1A | 26.15 ± 0.07 | 27.38 ± 0.22 | 26.11 ± 0.15 | 26.47 ± 0.08 | 25.45 ± 0.17 |
| | 26.63 ± 0.08 | 27.22 ± 0.10 | 25.78 ± 0.15 | 27.36 ± 0.23 | 26.00 ± 0.06 |
| | change 1.91/2.36 | change 66.72/5.02 | change 2.93/1.34 | change 4.11/1.84 | change 0.73/1.70 |
| ILB1 | 33.27 ± 0.68 | 33.75 ± 0.09 | 32.43 ± 0.27 | 30.29 ± 0.19 | 30.75 ± 0.66 |
| | 32.82 ± 0.22 | 31.27 ± 0.31 | 34.18 ± 0.22 | 30.37 ± 0.11 | 32.12 ± 0.37 |
| | change 1.00/1.08 | change 13.36/3.83 | change 12.38/2.05 | change 2.35/2.89 | change 1.29/1.61 |
| CXCL1 | 32.05 ± 0.34 | 33.22 ± 0.36 | 30.83 ± 0.38 | 30.32 ± 0.26 | 28.68 ± 0.39 |
| | 31.75 ± 0.38 | 32.35 ± 0.67 | 33.35 ± 0.13 | 31.22 ± 0.29 | 30.03 ± 0.21 |
| | change 1.11/1.12 | change 40.79/2.18 | change 21.11/3.05 | change 4.14/2.03 | change 1.27/1.77 |
| TNFAIP3 | 36.31 ± 0.61 | 37.18 ± 0.15 | 36.38 ± 0.07 | 37.87 ± 0.19 | 35.60 ± 0.81 |
| | 35.95 ± 0.30 | 37.31 ± 0.88 | 37.90 ± 0.42 | 39.18 ± 0.13 | 36.37 ± 0.46 |
| | change 1.06/1.74 | change 81.57/5.98 | change 10.56/1.59 | change 5.50/1.56 | change 0.85/1.52 |

^{a}C_{T} represents the cycle number at which a significant increase in fluorescence signal above a threshold signal (horizontal zero line) can first be detected.
^{b}X and SD, average and standard deviation values.
^{c}Using the comparative (ΔΔ C_{T}) method.
^{d}Internal control.

### EXAMPLE 2

### Prophetic Example of Detection of R. prowazekii Infection Using PBMC as Cell Source

As an illustration of the inventive method, R. prowazekii infection can be detected and diagnosed by measuring the modulation of specific genes by microarray analysis. The source of RNA for this analysis can be peripheral blood mononuclear cells (PBMC) Although the procedure is described here using PBMCs as an RNA source are, other purified cell populations, such as lymphocytes, can be utilized as well.

PBMCs are obtained from whole blood from healthy individuals by drawing the blood into cell preparation tubes containing anti-clotting agents, such as citrate. The tubes are then inverted 8 to 10 times and centrifuged at 1,500×g for 30 minutes at room temperature. Plasma is then removed and the PBMCs carefully removed. PBMCs enriched for monocytes by adjusting the PBMC populations to achieve a 4:1 ratio of lymphocytes. After washing the cells in phosphate buffered saline (PBS) the cells are suspended in RPMI 1640 media, supplemented with 2.5 mM L-glutamine, 25 mM HEPES and 20% fetal calf serum. The cells are then re-centrifuged and subsequently re-suspended in RPMI media until used.

The PBMCs are exposed to R. prowazekii for 30 minutes in 500 μl of RPMI. Some cells are also incubated without the addition of bacteria for use in the preparation of control RNA. After 30 minutes, the cells are washed with media and re-suspended in 48 ml of complete media (RPMI supplemented with supplemented with 2.5 mM L-glutamine, 25 mM HEPES and 20% fetal calf serum). Five ml of the re-suspended cells added to flasks containing 20 ml of the RPMI media supplemented with 2.5 mM L-glutamine, 25 mM HEPES and 20% fetal calf serum. At specific times after culturing the cells are scraped off and the RNA prepared utilizing the TRIzol® method (Invitrogen, Calsbad, Calif.). After preparation of RNA, the RNA was treated with DNAse to remove remaining amounts of contaminating DNA. The resulting RNA was stored at −80° C. until required.

Control and R. prowazekii RNA are reverse transcribed with oligo dT or random primers to synthesize cDNAs. The cDNA is then labeled with either Cy3 or Cy5 and commercial reference RNA is labeled with the dye not used in the labeling of sample cDNA. Labeled cDNA is permitted to hybridize at 42° C. overnight to glass chips containing cDNA gene sequences encoding host gene segments, including genes that are specifically modulated in response to R. prowazekii. In addition to possibly other sequences, the microarray chip should contain sequences represented in Table 2. Alternatively, detection of infection by R. prowazekii is possible by analysis of genes by a microarray containing only sequences encoding the genes OLIG2, POU3F4, ELK3, PPP1R14B, PRG1, CA2, BMI1, PLA2R1, HISTIH4C, CITED2 and DHRS9. The result obtained from this chip, may, however, be less definitive than utilizing a more complete gene panel as those listed in Table 2. To increase the speed of conduct of the assay, pre-made microarray chips containing the necessary host gene sequences can be made before hand.

As illustrated in Example 1, the cDNA clones are spotted onto poly-L-lysine-coated slides using an OmniGrid arrayer® (GeneMachines, San Carlos, Calif.). After hybridization, the bound, labeled cDNA is scanned and the image computer analyzed. An illustrative computer image analysis software is the GenePix® (Molecular Devices Corporation, Union City, Calif.). Normalization of induced expression, following R. prowazekii infection, is conducted by comparing expression of RNA from obtained PBMCs to control RNA from uninfected PBMCs and subtracting from the spot intensity the background intensity to produce a channel-specific value. Although other data analysis procedures are possible, a preferred embodiment is to converted the raw data into log 2 data. In this example, a change of ≧1.5 fold increase is scored as gene up-regulation and a change of ≦0.667 fold decrease expression is scored as down-regulation. However, the threshold change for gene modulation determination may vary depending on method of measurement and computer software used.

If the PBMCs had been obtained from presenting patients, early treatment, prior to that capable using currently available methods, can be initiated. Diagnosis is made by comparing and contrasting the gene modulation profile of the obtained PBMC with the expected gene modulation pattern following infection with R. prowazekii, shown in Table 2 or Table 3 and Table 4, if the microarray contained only those genes. An incomplete match of gene expression with the expected pattern, as in Table 2, 3 or 4, may indicate that an infection has occurred but is not R. prowazekii.

Despite the incomplete concordance between gene profiles from PBMCs obtained from patients and the anticipated gene expression profiles, the care provider may choose to initiate antibiotic treatment with further follow-up assays later. Follow-up, confirmatory diagnostic assays, such as qPCR or antibody-based assays for the detection of bacterial antigen, can be undertaken in order to give further assurance of infection and strain identification. Furthermore, additional assays, during the course of the disease, by microarray analysis or by other traditional diagnostic methods using fresh PBMCs, can be undertaken to monitor the disease progression and effectiveness of treatment.

### REFERENCES

1. More, J. B., C. E. Pedersen. (1980) The impact of rickettsial diseases on military operations. Mil. Med. 145: 780-785.
2. Patterson, K. D., (1993) Typhus and its control in Russia, 1870-1940. Medical History 37: 361-381.
3. Big, M.-L., B. LaScola, V. Roux, P. Brouqui, and D. Raoult. (1999) Isolation of Rickettsia prowazekii from blood by shell vial culture. J. Clin. Microbiol. 37: 3722-3724.
4. Raoult, D., J B. Ndihokubwayo, H. Tissot-Dupont, V. Roux, B. Faugere, R. Abegbinni, and R J. Birtles. (1998) Outbreak of epidemic typhus associated with trench fever in Burundi. Lancet 352:353-358.
5. Raoualt, D., V. Roux, J B. Ndihokubwayo, G. Bise, D. Baudon, G. Martet, and R. Birtles. (1997) Jail fever (epidemic typhus) outbreak in Burundi. Emerg. Infect. Dis., 3:357-359.
6. Raoult, D. R J Birtles, M Montoya, E Perez, H Tissot-Dupont, V Roux, and H. Guerra. (1999) Survey of three bacterial louse-associated diseases among rural Andean communities in Peru: Prevalence of epidemic typhus, trench fever, and relapsing fever. Clin. Infect. Dis. 29:434-436.
7. Health Aspects of Chemical and Biological Weapons. Geneva, Switzerland: World Health Organization, 1970: 98.
8. Weiss, E., and H. R. Dressler. (1962) Increased resistance to chloramphenicol in Rickettsia prowazekii with a note on failure to demonstrate genetic interaction among strains. J. Bacteriol. 83(2): 409-414.
9. Balayeva, N. M. O. M. Frolava, V. A. Genig, and V. N. Nikolskaya. (1985) Some biological properties of antibiotic resistant mutants of Rickettsia prowazekii strain E induced by nitroso-guanidine, p 85-91. In J. Kazar (ed.), Rickettsiae and rickettsial diseases. Publishing House of Slovak Academy of Sciences, Bratislava, Slovakia.
10. Rachek, L. I., A. M. Tucker, H. H. Winkler, and D. O. Wood. (1998) Transformation of Rickettsia prowazekii to Rifampin Resistance. J. Bact. 180:2118-2124.
11. Furuya, Y., Y. Yoshida, T. Katayama, F. Kawamori, S. Yamamoto, N. Ohashi, A. Kamura, A. Jr. Kawamura. (1991) Specific amplification of Rickettsia tsutsugamushi DNA from clinical specimen by polymerase chain reaction. J. Clin. Microbiol. 29:2628-2630.
12. Kelly, D J., G A. Dasch, T C. Chan, T. M. Ho. (1994) Detection and characterization of Rickettsia tsutsugamush (Rickettsiales: Rickettsiaceae) in infected Leptotrombidium fle,tcheri chiggers (Acari: Trombiculidae) with the polymerase chain reaction. J. Med. Etomol. 31:691-699.
13. Carl, M., C W. Tibbs, M E. Dobson, S F. Paparello, G A. Dasch. (1990) Diagnosis of acute typhus infection using the polymerase chain reaction. J. Infect. Dis. 161:791793.
14. Pradutkanchana, J., K. Silpapaojakul, H. Paxton, S. Pradutkanchana, D J. Kelly, D. Strickman. (1997) Comparative evaluation of four serodiagnostic tests for scrub typhus in Thailand. Trans. Roy. Soc. Trop. Med. Hyg. 91:425-428.
15. Eremeeva, M E., N M. Balayeva, D. Raoult. (1994) Serological response of patients suffering from primary and recrudescent typhus: comparison of complement fixation reaction, Weil-Felix test, microimmunofluorescence, and immunoblotting. Clin. Diagn. Lab. Immunol. 1:318-324.
16. Jett, M. Alterations in Gene Expression Show Unique Patterns in Response to Toxic Agents. 21st Army Science Conference, Proceedings 21:529-534.
17. Lockhart D J and E. A. Winzeler. (2000) Genomics, gene expression and DNA arrays. Nature. 405: 827-836.
18. Eckmann L, J. R. Smith, M. P. Housley, M. B. Dwinell and M. F. Kagnoff. J (2000) Analysis by high density cDNA arrays of altered gene expression in human intestinal epithelial cells in response to infection with the invasive enteric bacteria Salmonella. J. Biol. Chem. 275(19): 14084-14094.
19. Blader I J, I. D. Manger, and J. C. Boothroyd. (2001) Microarray analysis reveals previously unknown changes in Toxoplasma gondii-infected human cells. JBC. 276:24223-24231.
20. Ge, H., Chuang, Y.-Y. E., Zhao, S., Temenak, J. J., Ching, W.-M.(2004). Comparative genomics of Rickettsia prowazekii Madrid E and Breinl strains. J. Bacteriol 186 (2): 556-565.
21. Nau, G. J., J. F. L. (2002) Richmon, A. Schlesinger, E. G. Jennings, E. S. Lander and R. A. Young, Human macrophage activation programs induced by bacterial pathogens. Proc. Natl. Acad. Sci (USA) vol 99 (3): 1503-1508.

Having described the invention, one of skill in the art will appreciate in the claims that many modifications and variations of the present invention are possible in light of the above teachings. It is therefore, to be understood that, within the scope of the claims, the invention may be practiced otherwise than as specifically described.

What is claimed is:

1. A method for detecting Rickettsia prowazekii infection wherein said method is by analysis of host gene modulation in response to Rickettsia prowazekii infection comprising the steps:
a. determining a change in expression of genes from patient cells and;
b. comparing the expression of genes from said patient cells to an expected Rickettsia prowazekii induced or repressed gene expression pattern.

2. The method of claim 1, wherein said comparing of expression of genes from said patients is to said expected Rickettsia prowazekii induced gene expression pattern which comprises the genes: OLIG2, POU3F4, ELK3 and PPP1R14B and said expected repressed gene expression pattern comprising the genes: PRG1, CA2, BMI1, PLA2R1 HIST1H4C, CITED2 and DHRS9.

3. The method of claim 1, wherein said comparing of expression of genes from said patients is to said expected Rickettsia prowazekii induced gene expression pattern which comprises the genes: IL1B, IL1RL1, G1P3, IL8, MIP1B/CCL4L, CXCL1/GRO1, CXCL3/GRO3/MIP2B, CXCL13/BLC, CCL2/MCP1, CCL3/MIP1A, CCL20, ITGA2/CD49B, CDH2, CDH12, ROBO1, CAPN9, TNFAIP6, LPXN, PCDH8, APC, SGCD, COL2A1, PPL, C8orf1, ARPC2, SPRR1B, HPCA, AQP4, KCNS3, SLC25A10, CLIC4, ABCA1, APOH, EFEMP1, STC1, PLEK, S100A8, S100A9, S100A12, DSG2, IL7R, P2RY5, EDNRB, PTPN2, PTPRB, RARG, ACVR1, ENG, SERPINE2, KLRC3, FCER1A, LY64, IL27RA, KYNU, MAP3K71P2, PIK3R2, RGS1, PDE4B, ARF5, TM4SF3, LTBP2, CDC42, ARL4A, RAGE, GEM, HSPA8, DUSP6, ECT2, NFKB1A, NRIP1, TFAP2C, OLIG2, CREM, Ikaros, KLF2, POU3F4, IFI16, ELK3, TFEC, CXXC1, ZNF638, SNAPC3, TNFAIP3, TNFAIP8, SNCA, PRKR, PTEN, GADD45 alpha, PTHLH, CCNF, MTCP1, IGFBP3, INDO, AMPD3, USP12, USP32, UBE2B, SIAT8A, SIAT8E, SIAT1, ENTPD4, ACADL, PPP1R14B, COX8A, POLA2, ADH1B, ACSL1, CYP2C9, CYP1B1, TDO2, ENTPD4, B3GALT2, CD83, CYBB, DLX4, EGR2, MAFB, CDR2, C6orf142, DEAF1, RAE1, BM039, WBP1, MALAT1, PLAU, GJA7 and the said pattern of repressed genes which comprises the genes: RG1, CA2, BMI1, STAT5B, NCF4, PLA2R1, RPS15A, HIST1H4C, MYB, CITED2, DHRS9.

4. The method of claim 1 wherein said method of detecting Rickettsia prowazekii infection is used for the diagnosis of Rickettsia prowazekii infection early after infection.

5. The method of claim 1 wherein said patient cells are collected at various times from 1 to 14 days after infection and wherein said detection method is used to track the progression of said infection.

6. The method of claim 1 wherein said cells are peripheral blood mononuclear cells.

7. The method of claim 1 wherein said patient and control cells are a purified cell population selected from the group consisting of leukocytes, lymphocytes and macrophages.

8. The method of claim 1 wherein said change in expression of genes is determined by microarray analysis.

9. The method of claim 8, wherein said determination of said change in gene expression is by computer analysis.

10. The method of claim 1 wherein said change in expression of said genes is determined by enzyme-linked immunosorbent assay detection of protein products of said genes.

11. The method of claim 1 wherein said change in expression of said genes is by quantitative polymerase chain reaction.

12. The method of claim 8, wherein said change in expression of said genes is confirmed by the additional step of determining a change in expression of said genes by quantitative polymerase chain reaction.
